(19) United States
(12) Patent Application Publication
(10) Pub. No.: US 2025/0037840 A1
(43) Pub. Date: Jan. 30, 2025

(54) SYSTEMS AND METHODS FOR NATIVE DIRECT CONSOLE TO PATIENT DEVICE IMAGE SHARING

(71) Applicant: GE Precision Healthcare LLC, Waukesha, WI (US)

(72) Inventor: Alexander Roland Christian Aigner, Linz (AT)

(21) Appl. No.: 18/358,246

(22) Filed: Jul. 25, 2023

(51) Int. Cl. G16H 30/20; G06F 3/0484; G16H 10/60; H04W 12/08

(52) U.S. Cl. CPC G16H 30/20; G06F 3/0484; G16H 10/60; H04W 12/08; G06F 3/0482; G06K 7/1417; G06K 19/06037; G06K 19/06112

(57) ABSTRACT

A computer-implemented method for sharing ultrasound imaging data includes receiving, at a processor of a console of an ultrasound imaging system, a selection of one or more ultrasound images and/or cine clips of a region of a subject acquired by the ultrasound imaging system. The computer-implemented method also includes receiving, at the processor, a first input to start sharing the one or more ultrasound images and/or cine clips to a portable computing device associated with the subject. The portable computing device includes a camera. The computer-implemented method further includes initiating, via the processor, sharing of the one or more ultrasound images and/or cine clips directly from the console to the portable computing device via a device-to-device connection.

### BACKGROUND

The subject matter disclosed herein relates to medical imaging and, more particularly, systems and methods for native direct console to patient device image sharing.

An ultrasound device may be used for imaging targets such as organs and soft tissues in a human body, as well non-human targets. For example, an ultrasound device may be used for applications such as ultrasound/acoustic sensing, non-destructive evaluation (NDE), ultrasound therapy (e.g., High Intensity Focused Ultrasound (HIFU)), etc., in addition to ultrasound imaging of humans, animals, etc.

Ultrasound devices may use real time, non-invasive high frequency sound waves to produce a series of two-dimensional (2D) and/or three-dimensional (3D) images. The sound waves may be transmitted by a transmit transducer, and the reflections of the transmitted sound waves may be received by a receive transducer. The received sound waves may then be processed to display an image of the target. Sometimes patients want their images available to them digitally.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for sharing ultrasound imaging data is provided. The computer-implemented method includes receiving, at a processor of a console of an ultrasound imaging system, a selection of one or more ultrasound images and/or cine clips of a region of a subject acquired by the ultrasound imaging system. The computer-implemented method also includes receiving, at the processor, a first input to start sharing the one or more ultrasound images and/or cine clips to a portable computing device associated with the subject. The portable computing device includes a camera. The computer-implemented method further includes initiating, via the processor, sharing of the one or more ultrasound images and/or cine clips directly from the console to the portable computing device via a device-to-device connection.

In another embodiment, an ultrasound imaging system is provided. The ultrasound imaging system includes a display. The ultrasound imaging system also includes a memory encoding processor-executable routines. The ultrasound imaging system further includes a processor configured to access the memory and to execute the processor-executable routines, wherein the routines, when executed by the processor, cause the processor to perform actions. The actions include receiving a selection of one or more ultrasound images and/or cine clips of a region of a subject acquired by the ultrasound imaging system. The actions also include receiving a first input to start sharing the one or more ultrasound images and/or cine clips to a portable computing device associated with the subject. The portable computing device includes a camera. The actions further include initiating sharing of the one or more ultrasound images and/or cine clips directly from the ultrasound imaging system to the portable computing device via a device-to-device connection.

In a further embodiment, a non-transitory computer-readable medium, the computer-readable medium including processor-executable code that when executed by a processor, causes the processor to perform actions. The actions include receiving a selection of one or more ultrasound images and/or cine clips of a region of a subject acquired by the ultrasound imaging system. The actions also include receiving a first input to start sharing the one or more ultrasound images and/or cine clips to a portable computing device associated with the subject. The portable computing device includes a camera. The actions further include initiating sharing of the one or more ultrasound images and/or cine clips directly from the ultrasound imaging system to the portable computing device via a device-to-device connection.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a block diagram of an ultrasound system, in accordance with aspects of the present disclosure;

FIG. 2 is a flow chart of a method for sharing ultrasound imaging data, in accordance with aspects of the present disclosure;

FIG. 3 is a flow chart of a method for sharing ultrasound imaging data (e.g., utilizing quick response codes), in accordance with aspects of the present disclosure;

FIG. 4 is a flow chart of a method for sharing ultrasound imaging data (e.g., from the perspective of a portable computing device), in accordance with aspects of the present disclosure;

FIG. 5 is a schematic diagram of a process for sharing ultrasound imaging data directly between an ultrasound system and a portable computing device of a patient, in accordance with aspects of the present disclosure;

FIG. 6 is a schematic diagram of a user interface on a display of an ultrasound system for initiating sharing of ultrasound imaging data, in accordance with aspects of the present disclosure;

FIG. 7 is a schematic diagram of a user interface on a display of an ultrasound system once sharing of ultrasound imaging data has begun (e.g., having button for stopping sharing), in accordance with aspects of the present disclosure;

FIG. 8 is a schematic diagram of a user interface on a display of an ultrasound system once sharing of ultrasound imaging data has begun (e.g., having a timer for sharing), in accordance with aspects of the present disclosure;

FIG. 9 is a schematic diagram of a user interface on a display of an ultrasound system for initiating sharing of ultrasound imaging data (e.g. having selection for ending sharing), in accordance with aspects of the present disclosure; and

FIG. 10 is a schematic diagram of an example processor platform for a portable computing device to be utilized with the disclosed techniques.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

As used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

The present disclosure provides for systems and methods for sharing ultrasound imaging data directly from an ultrasound imaging system to a patient's device (e.g., portable computing device). In particular, the disclosed systems and methods include receiving, at a processor of a console of an ultrasound imaging system, a selection of one or more ultrasound images and/or cine (e.g., video) clips of a region of a subject (e.g., patient) acquired by the ultrasound imaging system. The disclosed systems and methods also include receiving, at the processor, a first input to start sharing the one or more ultrasound images and/or cine clips to a portable computing device associated with the subject (e.g., the patient's device or a device of the patient's guardian if the patient is a child). The portable computing device includes a camera. The disclosed systems and methods further include initiating, via the processor, sharing of the one or more ultrasound images and/or cine clips directly from the console to the portable computing device via a device-to-device connection (e.g., machine-to-machine connection).

The disclosed systems and methods include ceasing, via the processor, the sharing of the one or more ultrasound images and/or cine clips to the portable computing device. In certain embodiments, ceasing the sharing of the one or more ultrasound images and/or cine clips includes receiving a second input, at the processor, to cease the sharing of the one or more ultrasound images and/or cine clips. In certain embodiments, ceasing the sharing of the one or more ultrasound images and/or cine clips includes automatically ceasing, via the processor, the sharing of the one or more ultrasound images and/or cine clips after a fixed amount of time passes after initiation of the sharing of the one or more ultrasound images and/or cine clips.

The disclosed systems and methods include initiating, via the processor, a secure Wi-Fi hotspot for a single inbound connection upon receiving the first input. The disclosed systems and methods also include causing, via the processor, display of a Wi-Fi access quick response (QR) code on a display of the ultrasound imaging system. The Wi-Fi access quick response code is natively configured to work with an operating system of the portable computing device. The disclosed systems and methods further include detecting, via the processor, a connection to the secure Wi-Fi hotspot by the portable computing device when the portable computing device utilizes the Wi-Fi access quick response code. The disclosed systems and methods yet further include initiating, via the processor, a content file server upon detecting the connection to the secure Wi-Fi hotspot. The content file server is restricted to only access the one or more ultrasound images and/or cine clips that were selected. The disclosed systems and methods still further include causing, via the processor, display of a content uniform resource locator (URL) quick response code on the display of the ultrasound imaging system, wherein the content uniform resource locator quick response code is natively configured to work with an operating system of the portable computing device, and the content uniform resource locator quick response code is configured to provide the portable computing device access to the one or more ultrasound images and/or cine clips when utilized by the portable computing device.

In the disclosed embodiments, direct device (e.g., ultrasound imaging system or console) to device (e.g., patient device) connectivity for downloading or sharing images and/or cines on the patient device is provided in a fast, secure, and easy process. The software for this is native (e.g., built into) to ultrasound imaging system. The disclosed embodiments also enable patients to receive their ultrasound images or cines directly after the scan on their device (e.g., smartphone, tablet, etc.). The disclosed embodiments further provide the images or cines without any preparations needed on the patient's side (e.g., no dedicated application is needed, ultrasound console works out-of-the box with the operating system of the patient's device, machine-to-machine connection utilized with no Internet or cloud connectivity involved). The disclosed embodiments even further enable image sharing to occur in areas without Internet access on the patient's device (e.g., lower levels of hospitals, rural areas, etc.). The disclosed embodiments yet further include enabling a patient to instantly share or postprocess the image further on their device. Although the disclosed techniques are discussed in the context of analysis of ultrasound images, the disclosed techniques may be utilized on any medical images (e.g., derived from conventional X-ray imaging, computed tomography imaging, nuclear medicine imaging, magnetic resonance imaging, or other type of imaging modality).

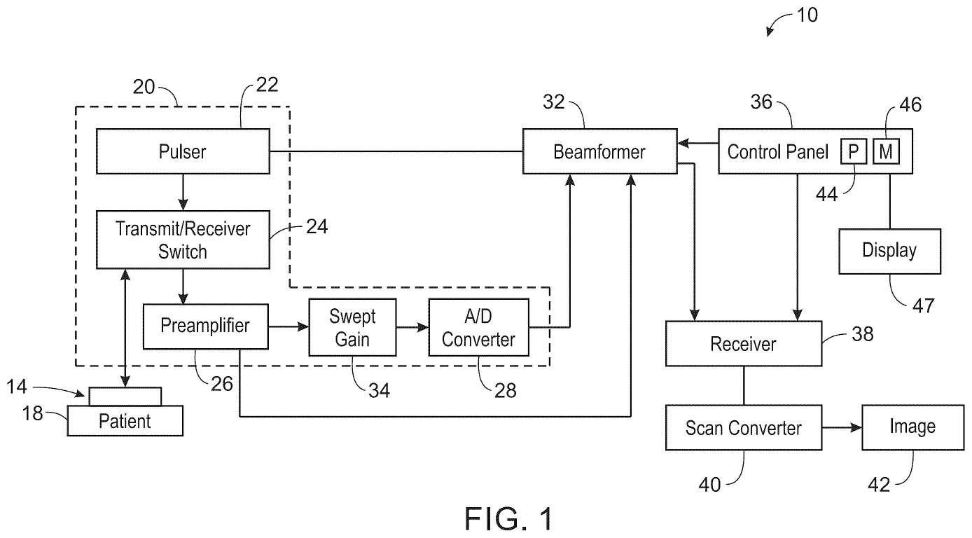

With the preceding in mind, and by way of providing useful context, FIG. 1 depicts a high-level view of components of an ultrasound system 10 (e.g., ultrasound imaging system) that may be employed in accordance with the present approach. The illustrated ultrasound system 10 includes a transducer array 14 having transducer elements suitable for contact with a subject or patient 18 during an imaging procedure. The transducer array 14 may be configured as a two-way transducer capable of transmitting ultrasound waves into and receiving such energy from the subject or patient 18. In such an implementation, in the transmission mode the transducer array elements convert electrical energy into ultrasound waves and transmit it into the patient 18. In reception mode, the transducer array elements convert the ultrasound energy received from the patient 18 (backscattered waves) into electrical signals.

Each transducer element is associated with respective transducer circuitry, which may be provided as one or more application specific integrated circuits (ASICs) 20, which may be present in a probe or probe handle. That is, each transducer element in the array 14 is electrically connected to a respective pulser 22, transmit/receive switch 24, preamplifier 26, swept gain 34, and/or analog to digital (A/D) converter 28 provided as part of or on an ASIC 20. In other implementations, this arrangement may be simplified or otherwise changed. For example, components shown in the circuitry 20 may be provided upstream or downstream of the depicted arrangement, however, the basic functionality depicted will typically still be provided for each transducer element. In the depicted example, the referenced circuit functions are conceptualized as being implemented on a single ASIC 20 (denoted by dashed line), however it may be appreciated that some or all of these functions may be provided on the same or different integrated circuits.

Also depicted in FIG. 1, a variety of other imaging components are provided to enable image formation with the ultrasound system 10. Specifically, the depicted example of an ultrasound system 10 also includes a beam former 32, a control panel 36 (e.g., console), a receiver 38, and a scan converter 40 that cooperate with the transducer circuitry to produce an image or series of images 42 that may be stored and/or displayed to an operator or otherwise processed as discussed herein. The transducer array 14 may be communicate the ultrasound data to the beam-former via a wired connection or wireless connection (e.g., via a wireless communication unit that is part of the transducer array that communicates over a Wi-Fi network, utilizing Bluetooth® technique, or some other manner).

A processing component 44 (e.g., a microprocessor or processing circuitry) and a memory 46 of the system 10, such as may be present control panel 36, may be used to execute stored routines for processing the acquired ultrasound signals to generate meaningful images and/or motion frames, which may be displayed on a display 47 of the ultrasound system 10. The processing component 44 may receive a selection of one or more ultrasound images and/or cine (e.g., video) clips of a region of a subject (e.g., patient) acquired by the ultrasound system 10. The processing component 44 may also receive a first input to start sharing the one or more ultrasound images and/or cine clips to a portable computing device associated with the subject (e.g., the patient's device or a device of the patient's guardian if the patient is a child). The portable computing device includes a camera. The processing component 44 may further initiate sharing of the one or more ultrasound images and/or cine clips directly from the console of the ultrasound system 10 to the portable computing device via a device-to-device connection (e.g., machine-to-machine connection).

The processing component 44 may cease the sharing of the one or more ultrasound images and/or cine clips to the portable computing device. In certain embodiments, ceasing the sharing of the one or more ultrasound images and/or cine clips includes the processing component 44 receiving a second input to cease the sharing of the one or more ultrasound images and/or cine clips. In certain embodiments, ceasing the sharing of the one or more ultrasound images and/or cine clips includes the processing component 44 automatically ceasing the sharing of the one or more ultrasound images and/or cine clips after a fixed amount of time passes after initiation of the sharing of the one or more ultrasound images and/or cine clips.

The processing component may initiate a secure Wi-Fi hotspot for a single inbound connection upon receiving the first input. The processing component 44 may also cause display of a Wi-Fi access quick response code on the display 47 of the ultrasound system 10. The Wi-Fi access quick response code is natively configured to work with an operating system of the portable computing device. The processing component 44 may further detect a connection to the secure Wi-Fi hotspot by the portable computing device when the portable computing device utilizes the Wi-Fi access quick response code. The processing component 44 may yet further initiate a content file server upon detecting the connection to the secure Wi-Fi hotspot. The content file server is restricted to only access the one or more ultrasound images and/or cine clips that were selected. The processing component 44 still further may cause display of a content uniform resource locator quick response code on the display 47 of the ultrasound system 10, wherein the content uniform resource locator quick response code is natively configured to work with an operating system of the portable computing device, and the content uniform resource locator quick response code is configured to provide the portable computing device access to the one or more ultrasound images and/or cine clips when utilized by the portable computing device.

Ultrasound information may be processed by other or different mode-related modules (e.g., B-mode, Color Doppler, power Doppler, M-mode, spectral Doppler anatomical M-mode, strain, strain rate, and the like) to form 2D or 3D data sets of image frames and the like. For example, one or more modules may generate B-mode, color Doppler, power Doppler, M-mode, anatomical M-mode, strain, strain rate, spectral Doppler image frames and combinations thereof, and the like. The image frames are stored and timing information indicating a time at which the image frame was acquired in memory may be recorded with each image frame. The modules may include, for example, a scan conversion module to perform scan conversion operations to convert the image frames from Polar to Cartesian coordinates. A video processor module may be provided that reads the image frames from a memory and displays the image frames in real time while a procedure is being carried out on a patient. A video processor module may store the image frames in an image memory, from which the images are read and displayed. The ultrasound system 10 shown may comprise a console system, or a portable system, such as a hand-held or laptop-type system.

The ultrasound system 10 may be operable to continuously acquire ultrasound scan data at a frame rate that is suitable for the imaging situation in question. Typical frame rates may range from 20-120 but may be lower or higher. The acquired ultrasound scan data may be displayed on the display 47 at a display-rate that can be the same as the frame rate, or slower or faster. An image buffer may be included for storing processed frames of acquired ultrasound scan data that are not scheduled to be displayed immediately. Preferably, the image buffer is of sufficient capacity to store at least several minutes worth of frames of ultrasound scan data. The frames of ultrasound scan data are stored in a manner to facilitate retrieval thereof according to its order or time of acquisition. The image buffer may be embodied as any known data storage medium.

The display 47 may be any device capable of communicating visual information to a user. For example, the display 47 may include a liquid crystal display, a light emitting diode display, and/or any suitable display or displays. The display 47 can be operable to present ultrasound images and/or any suitable information.

Components of the ultrasound system 10 may be implemented in software, hardware, firmware, and/or the like. The various components of the ultrasound system 10 may be communicatively linked. Components of the ultrasound may be implemented separately and/or integrated in various forms.

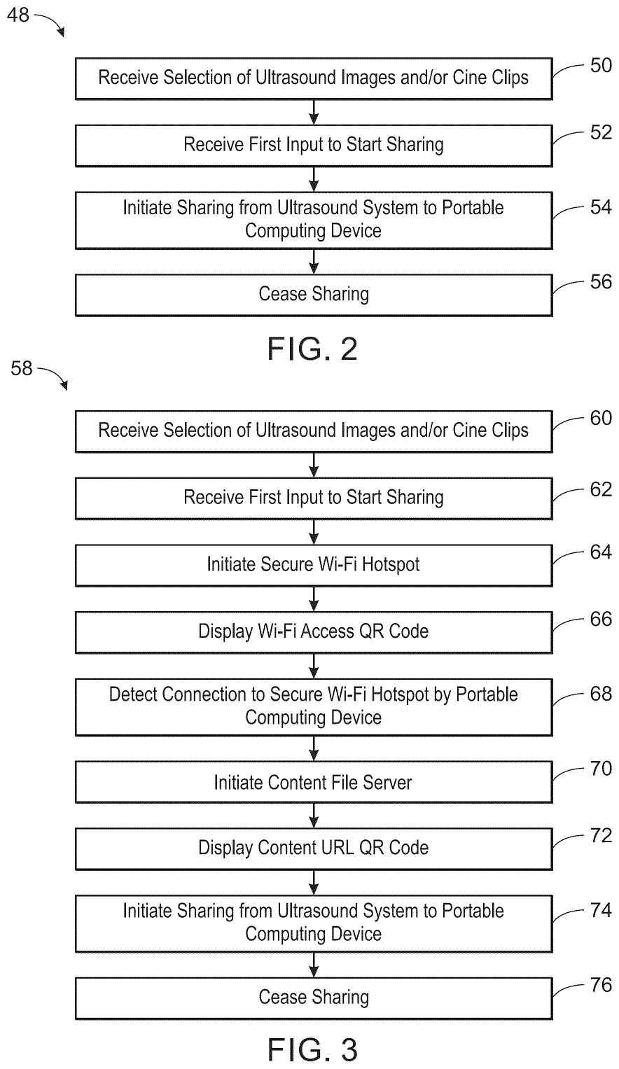

FIG. 2 is a flow chart of a method 48 for sharing ultrasound imaging data. One or more steps of the method 48 may be performed by processing circuitry of the ultrasound system 10 in FIG. 1.

The method 48 includes receiving a selection of one or more ultrasound images and/or cine clips of a region of a subject acquired by the ultrasound system 10 (block 50). The selection of the ultrasound images and/or cine clips may be manually made by the operator via an input device on the console of the ultrasound system 10. A plurality of ultrasound images and/or cine clips may be displayed on a display of the ultrasound system. The images or cine clips that are may be marked via an indicator (e.g., check mark) to indicate their selection from among the plurality of ultrasound images and/or cine clips.

The method 48 also includes receiving a first input to start sharing the one or more ultrasound images and/or cine clips to a portable computing device (e.g., smartphone, tablet, etc.) associated with the subject (e.g., patient's own portable computing device or the portable computing device of a guardian when the patient is a child) (block 52). A button may be presented for selection on a user interface on the display of the ultrasound system 10 to start sharing the selected ultrasound images and/or cine clips. The portable computing device includes a camera. The camera enables the portable computing device to scan quick response codes (via the native camera application on the portable computing device) to facilitate the sharing of the selected ultrasound images and/or cine clips.

The method 48 further includes initiating sharing of the selected one or more ultrasound images and/or cine clips directly from the ultrasound system 10 (e.g., ultrasound console) to the portable computing device via a device-to-device connection (block 54). The sharing over this device-to-device connection occurs without any Internet or cloud connectivity involved. In particular, the direct connection and content sharing occurs without utilizing the clinical network or internet or any other network. Only the native operating system functionality of portable computing device is utilized with no preparation needed.

The method 48 still further includes ceasing the sharing of the selected ultrasound images and/or cine clips to the portable computing device (block 56). In certain embodiments, ceasing the sharing of the selected ultrasound images and/or cine clips includes receiving a second input to cease the sharing of the selected ultrasound images and/or cine clips. For example, a button may be presented on a user interface on the display of the ultrasound system 10 for selection by the operator to cease the sharing. In certain embodiments, ceasing the sharing of the one or more ultrasound images and/or cine clips includes automatically ceasing the sharing of the selected ultrasound images and/or cine clips after a fixed amount of time passes after initiation of the sharing of the selected ultrasound images and/or cine clips. In certain embodiments, the amount of time allowed for sharing may be adjusted based on the amount of data shared.

FIG. 3 is a flow chart of a method 58 for sharing ultrasound imaging data (e.g., utilizing quick response codes). One or more steps of the method 58 may be performed by processing circuitry of the ultrasound system 10 in FIG. 1.

The method 58 includes receiving a selection of one or more ultrasound images and/or cine clips of a region of a subject acquired by the ultrasound system 10 (block 60). The selection of the ultrasound images and/or cine clips may be manually made by the operator via an input device on the console of the ultrasound system 10. A plurality of ultrasound images and/or cine clips may be displayed on a display of the ultrasound system. The images or cine clips that are selected may be marked via an indicator (e.g., check mark) to indicate their selection from among the plurality of ultrasound images and/or cine clips.

The method 58 also includes receiving a first input to start sharing the one or more ultrasound images and/or cine clips to a portable computing device (e.g., smartphone, tablet, etc.) associated with the subject (e.g., patient's own portable computing device or the portable computing device of a guardian when the patient is a child) (block 62). A button may be presented for selection on a user interface on the display of the ultrasound system 10 to start sharing the selected ultrasound images and/or cine clips. The portable computing device includes a camera. The camera enables the portable computing device to scan quick response codes (via the native camera application on the portable computing device) to facilitate the sharing of the selected ultrasound images and/or cine clips.

The method 58 further includes initiating a secure Wi-Fi hotspot upon receiving the first input (block 64). The Wi-Fi hotspot is limited to a single inbound connection. The Wi-Fi hotspot may utilize the Wi-Fi Protected Access 2 (WPA2) or its newest iteration as the encryption standard and pre-shared key (PSK) and advanced encryption standard (AES) or its newest iteration as the encryption protocol. The WPA2-PSK (AES) ensures the security of information during the sharing process. The access-password for the Wi-Fi hotspot is randomized utilizing a cryptographically secure pseudorandom number generator (CSPRNG) each time it is initiated. The Wi-Fi hotspot also avoids sharing via the internet or a wired connection.

The method 58 still further includes causing display of a Wi-Fi access quick response code on a display (e.g., display 47 in FIG. 1) of the ultrasound system 10 (block 66). The Wi-Fi access quick response code is natively configured to work with an operating system of the portable computing device. The Wi-Fi access quick response code may be scanned by the camera on the portable computing device utilizing the native camera application on the portable computing device and accessed via a single click by the user (e.g., patient) of the portable computing device. Scanning of the Wi-Fi access quick response code enables detection of the Wi-Fi access credentials. In certain embodiments, access to the Wi-Fi hotspot may occur automatically upon scanning the Wi-Fi access quick response code. Whether access involves a click by the user or is automatic may be adjustable (depending on the portable computing device). The method 58 yet further includes detecting a connection to the secure Wi-Fi hotspot by the portable computing device when the portable computing device utilizes the Wi-Fi access quick response code (block 68).

The method 58 even further includes initiating a content file server (or web server) upon detecting the connection to the secure Wi-Fi hotspot by the portable computing device (block 70). The content file server is restricted to only access the one or more ultrasound images and/or cine clips that were selected. The content file server only listens on the Wi-Fi interface. There is no server-side scripting with the content filer server. Instead, there is only static content. The content file server is hardened in accordance with the guidelines of the respective server software.

The method 58 yet further includes causing display of a content uniform resource locator quick response code on the display (e.g., display 47 in FIG. 1) of the ultrasound system 10 (block 72). The content uniform resource locator quick response code is natively configured to work with an operating system of the portable computing device. Also, the content uniform resource locator quick response code is configured to provide the portable computing device access to the one or more ultrasound images and/or cine clips. The content uniform resource locator quick response code may be scanned by the camera on the portable computing device utilizing the native camera application on the portable computing device and accessed via a single click by the user (e.g., patient) of the portable computing device. Scanning of the content uniform resource locator quick response code enables detection of the content uniform resource locator. In certain embodiments, access to the content uniform resource locator may occur automatically upon scanning the content uniform resource locator quick response code. Whether access involves a click by the user or is automatic may be adjustable (depending on the portable computing device). In certain embodiments, the content uniform resource locator may be provided to the portable computing device and opened via a browser on the portable computing device to view the selected ultrasound images and/or cine clips on a display of the portable computing device. In certain embodiments, the selected ultrasound images and/or cine clips may be directly shown (without having to open the content uniform resource locator via browser) on the display of the portable computing device.

The method 58 further includes initiating sharing of the selected one or more ultrasound images and/or cine clips directly from the ultrasound system 10 (e.g., ultrasound console) to the portable computing device via a device-to-device connection (block 74). The sharing over this device-to-device connection occurs without any Internet or cloud connectivity involved. In particular, the direct connection and content sharing occurs without utilizing the clinical network or internet or any other network. Only the native operating system functionality of portable computing device is utilized with no preparation needed.

The method 58 still further includes ceasing or stopping the sharing of the selected ultrasound images and/or cine clips to the portable computing device (block 76). In certain embodiments, ceasing the sharing of the selected ultrasound images and/or cine clips includes receiving a second input to cease the sharing of the selected ultrasound images and/or cine clips. For example, a button may be presented on a user interface on the display of the ultrasound system 10 for selection by the operator to cease the sharing. In certain embodiments, ceasing the sharing of the one or more ultrasound images and/or cine clips includes automatically ceasing the sharing of the selected ultrasound images and/or cine clips after a fixed amount of time passes after initiation of the sharing of the selected ultrasound images and/or cine clips. In certain embodiments, the amount of time allowed for sharing may be adjusted based on the amount of data shared. Ceasing the sharing also stops both the content file server and the Wi-Fi hot spot.

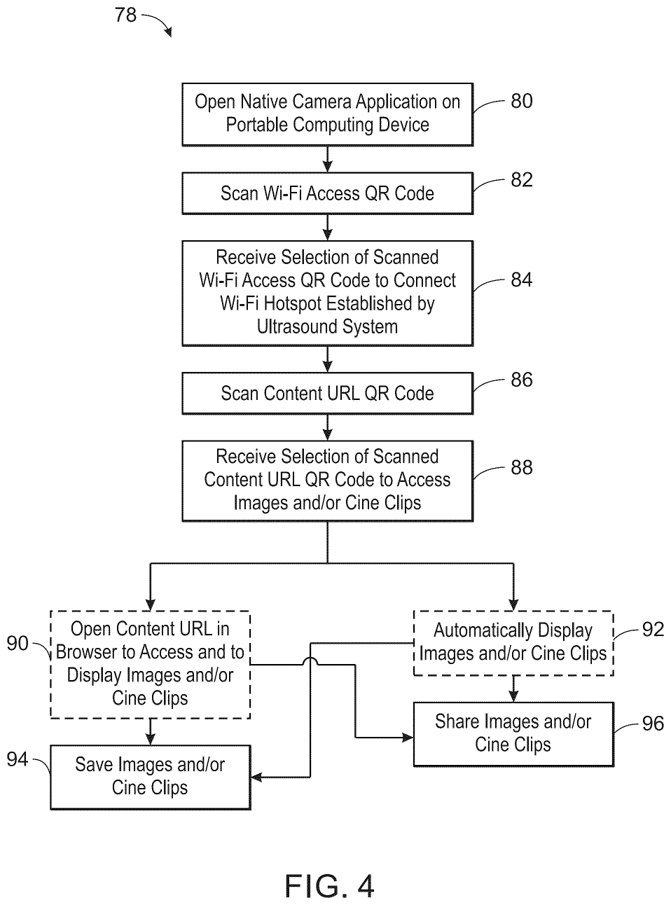

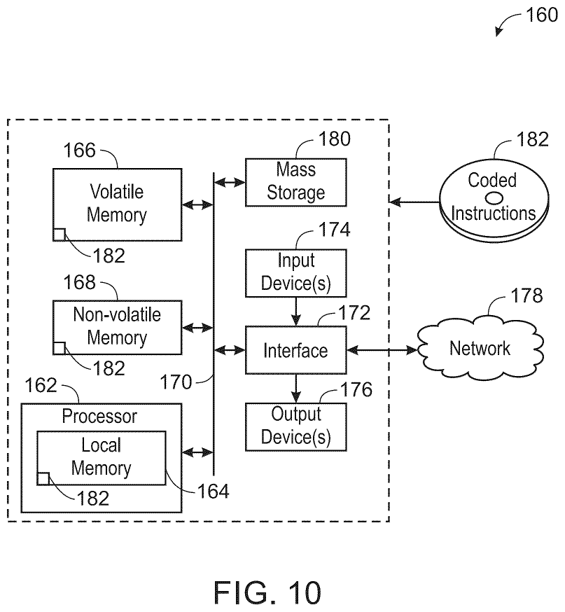

FIG. 4 is a flow chart of a method 78 for sharing ultrasound imaging data (e.g., from the perspective of a portable computing device). One or more steps of the method 78 may be performed by processing circuitry of the portable computing device in FIG. 10.

The method 78 includes opening a native camera application on a portable computing device (e.g., smartphone, tablet, etc.) having a camera (block 80). The portable computing device is associated with the subject (e.g., patient's own portable computing device or the portable computing device of a guardian when the patient is a child) having a region of interest scanned by an ultrasound system (e.g., ultrasound system 10 in FIG. 1). The method 80 also includes scanning a Wi-Fi access quick response code on a display (e.g., display 47 in FIG. 1) of the ultrasound system 10 (block 82). Scanning the Wi-Fi access quick response code enables detection of Wi-Fi access credentials of a Wi-Fi hotspot established by the ultrasound system 10. The method 78 further includes receiving a selection (e.g., a single click) of the scanned Wi-Fi access quick response code to connect to a secure Wi-Fi hotspot established by the ultrasound system 10 (block 84). In certain embodiments, access to the Wi-Fi hotspot may occur automatically upon scanning the Wi-Fi access quick response code. Whether access involves a click by the user or is automatic may be adjustable (depending on the portable computing device).

The method 78 still further includes scanning a content uniform resource locator quick response code on the display of the ultrasound system 10 (block 86). Scanning the content uniform resource locator quick response code enables detection of a content uniform resource locator for a content file server. The content file server includes selected ultrasound images and/or cine clips of a region of interest of the subject acquired by the ultrasound system 10. The method 78 also includes receiving a selection (e.g., a single click) of the scanned content uniform resource locator quick response code to access the selected ultrasound images and/or cine clips (block 88). In certain embodiments, access to the content uniform resource locator may occur automatically upon scanning the content uniform resource locator quick response code. Whether access involves a click by the user or is automatic may be adjustable (depending on the portable computing device). In certain embodiments, the method 78 includes opening the content uniform resource locator in a browser to access and display on the display of the portable computing device the selected ultrasound images and/or cine clips (block 90). In certain embodiments, the method 78 includes automatically displaying the selected ultrasound images and/or cine clips on the display of the portable computing device (block 92).

In certain embodiments, the method 78 includes saving the one or more ultrasound images and/or cine clips to the portable computing device (e.g., in the image gallery) utilizing the native operating system of the portable computing device (block 94). In certain embodiments, the method includes sharing one or more ultrasound images and/or cine clips from the portable computing device (e.g., via text, via email, etc.) (block 96).

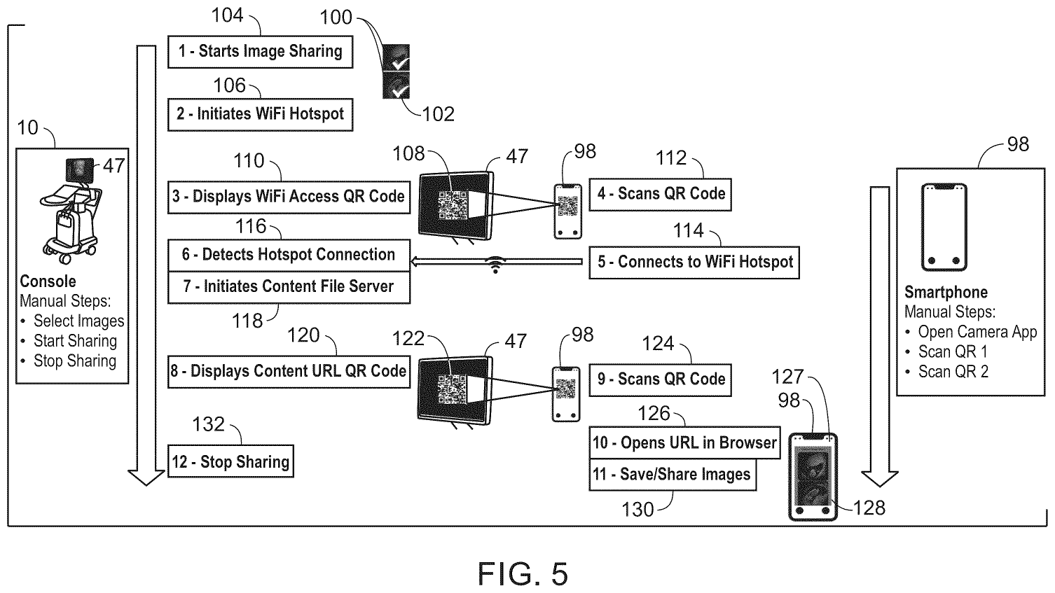

FIG. 5 is a schematic diagram of a process for sharing ultrasound imaging data directly between the ultrasound system 10 and a portable computing device 98 (e.g., smartphone, tablet, etc.) of a patient scanned by the ultrasound system 10 incorporating the methods 48, 58, and 78 in FIGS. 2-4, respectively. As depicted in FIG. 5, the portable computing device 98 is a smartphone. In certain embodiments, the portable computing device 98 may belong to a guardian of the patient (e.g., when the patient is a child). After an imaging session with the ultrasound system 10, the image sharing process begins with receiving a selection of one or more ultrasound images and/or cine clips of a region of a subject acquired by the ultrasound system 10. The selection of the ultrasound images and/or cine clips may be manually made by the operator via an input device on the console of the ultrasound system 10. A plurality of ultrasound images and/or cine clips may be displayed on the display 47 of the ultrasound system 10. The images or cine clips 102 that are selected may be marked via an indicator 104 (e.g., check mark) to indicate their selection from among the plurality of ultrasound images and/or cine clips.

The process also includes (as indicated by reference numeral 104) receiving a first input (on the console of the ultrasound system 10) to start sharing the one or more ultrasound images and/or cine clips to a portable computing device 98 (e.g., smartphone, tablet, etc.) associated with the subject (e.g., patient's own portable computing device 98 or the portable computing device 98 of a guardian when the patient is a child). A button may be presented for selection on a user interface on the display 47 of the ultrasound system 10 to start sharing the selected ultrasound images and/or cine clips. The portable computing device 98 includes a camera. The camera enables the portable computing device 98 to scan quick response codes (via the native camera application on the portable computing device 98) to facilitate the sharing of the selected ultrasound images and/or cine clips.

The process further includes initiating (via the ultrasound system 10) a secure Wi-Fi hotspot upon receiving the first input as indicated by reference numeral 106. The Wi-Fi hotspot is limited to a single inbound connection. The Wi-Fi hotspot may utilize the Wi-Fi Protected Access 2 (WPA2) or its newest iteration as the encryption standard and pre-shared key (PSK) and advanced encryption standard (AES) or its newest iteration as the encryption protocol. The WPA2-PSK (AES) ensures the security of information during the sharing process. The access-password for the Wi-Fi hotspot is randomized utilizing a cryptographically secure pseudorandom number generator (CSPRNG) each time it is initiated. The Wi-Fi hotspot also avoids sharing via the internet or a wired connection.

The process still further includes causing display of a Wi-Fi access quick response code 108 on the display 47 of the ultrasound system 10 as indicated by reference numeral 110. The Wi-Fi access quick response code 108 is natively configured to work with an operating system of the portable computing device 98.

The process includes opening a native camera application on the portable computing device 98 having a camera. The process also includes (as indicated by reference numeral 112) scanning the Wi-Fi access quick response code 108 on the display 47 of the ultrasound system 10. Scanning the Wi-Fi access quick response code enables detection of Wi-Fi access credentials of the Wi-Fi hotspot established by the ultrasound system 10. The process further includes receiving a selection (e.g., a single click) of the scanned Wi-Fi access quick response code to connect to a secure Wi-Fi hotspot established by the ultrasound system 10 as indicated by reference numeral 114. In certain embodiments, access to the Wi-Fi hotspot may occur automatically upon scanning the Wi-Fi access quick response code 108. Whether access involves a click by the user or is automatic may be adjustable (depending on the portable computing device 98). The process yet further includes detecting a connection to the secure Wi-Fi hotspot by the portable computing device 98 when the portable computing device 98 utilizes the Wi-Fi access quick response code 108 as indicated by reference numeral 116.

The process even further includes initiating (via the ultrasound system 10) a content file server (or web server) upon detecting the connection to the secure Wi-Fi hotspot by the portable computing device 98 as indicated by reference numeral 118. The content file server is restricted to only access the one or more ultrasound images and/or cine clips that were selected. The content file server only listens on the Wi-Fi interface. There is no server-side scripting with the content filer server. Instead, there is only static content. The content file server is hardened in accordance with the guidelines of the respective server software.

The process yet further includes (as indicated by reference numeral 120) causing display of a content uniform resource locator quick response code 122 on the display 47 of the ultrasound system 10. The content uniform resource locator quick response code 122 is natively configured to work with an operating system of the portable computing device 98. Also, the content uniform resource locator quick response code 122 is configured to provide the portable computing device 98 access to the one or more ultrasound images and/or cine clips.

The process still further includes (as indicate by reference numeral 124) scanning the content uniform resource locator quick response code 122 on the display 47 of the ultrasound system 10. Scanning the content uniform resource locator quick response code 122 enables detection of a content uniform resource locator for a content file server. The content file server includes selected ultrasound images and/or cine clips of a region of interest of the subject acquired by the ultrasound system 10. The process also includes receiving a selection (e.g., a single click) of the scanned content uniform resource locator quick response code 122 to access the selected ultrasound images and/or cine clips. In certain embodiments, access to the content uniform resource locator may occur automatically upon scanning the content uniform resource locator quick response code 122. Whether access involves a click by the user or is automatic may be adjustable (depending on the portable computing device 98).

In certain embodiments, the process includes (as indicated by reference numeral 126) opening the content uniform resource locator in a browser to access and display on the display 127 of the portable computing device 98 the selected ultrasound images and/or cine clips 128. In certain embodiments, the process includes automatically displaying the selected ultrasound images and/or cine clips 128 on the display 127 of the portable computing device 98. The sharing over this device-to-device connection occurs without any Internet or cloud connectivity involved. In particular, the direct connection and content sharing occurs without utilizing the clinical network or internet or any other network. Only the native operating system functionality of portable computing device 98 is utilized with no preparation needed.

In certain embodiments, the process includes (as indicated by reference numeral 130) saving the one or more ultrasound images and/or cine clips to the portable computing device 98 (e.g., in the image gallery) utilizing the native operating system of the portable computing device 98. In certain embodiments, the process includes (as indicated by reference numeral 130) sharing one or more ultrasound images and/or cine clips from the portable computing device (e.g., via text, via email, etc.).

The process still further includes ceasing or stopping the sharing of the selected ultrasound images and/or cine clips from the ultrasound system 10 to the portable computing device as indicated by reference numeral 132. In certain embodiments, ceasing the sharing of the selected ultrasound images and/or cine clips includes receiving a second input to cease the sharing of the selected ultrasound images and/or cine clips. For example, a button may be presented on a user interface on the display 47 of the ultrasound system 10 for selection by the operator to cease the sharing. In certain embodiments, ceasing the sharing of the one or more ultrasound images and/or cine clips includes automatically ceasing the sharing of the selected ultrasound images and/or cine clips after a fixed amount of time passes after initiation of the sharing of the selected ultrasound images and/or cine clips. In certain embodiments, the amount of time allowed for sharing may be adjusted based on the amount of data shared. Ceasing the sharing also stops both the content file server and the Wi-Fi hot spot.

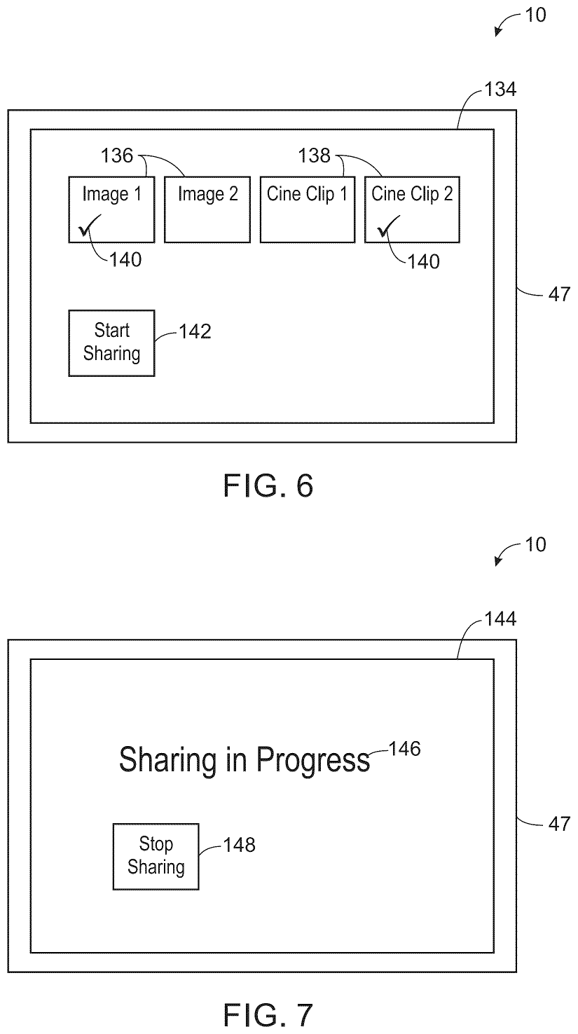

FIG. 6 is a schematic diagram of a user interface 134 on the display 47 of the ultrasound system 10 for initiating sharing of ultrasound imaging data. As depicted in FIG. 6, a plurality of ultrasound images 136 and cine clips 138 are displayed on the user interface 134 for selection by the operator of the ultrasound system 10. In certain embodiments, the plurality of ultrasound images 136 and cine clips 138 are displayed as thumbnails. Those ultrasound images 136 and cine clips selected (e.g., via a console of the ultrasound system 10) for direct sharing from the ultrasound system 10 to a portable computing device of the patient just scanned with the ultrasound system (or a guardian of the patient when the patient is a child) are marked with an indicator 140. As depicted, the user interface 134 includes a button 142 for selection to initiate the direct sharing of the selected ultrasound images 136 and cine clips 138. Upon the operator of the ultrasound system 10 selecting or clicking the button 142 the process for sharing the selected ultrasound images 136 and cine clips 138 begins.

FIG. 7 is a schematic diagram of a user interface 144 on the display 47 of the ultrasound system 10 once sharing of ultrasound imaging data has begun (e.g., having button for stopping sharing). In certain embodiments, during sharing of selected ultrasound images and/cine clips an indicator 146 (e.g., text) may be provided on the user interface 144 that sharing the selected ultrasound images and/or cine clips are being shared (e.g., to the portable computing device of the patient just scanned by the ultrasound system 10 or a guardian of the patient when the patient is a child). As depicted, the user interface 144 includes a button 148 for selection to cease or stop the direct sharing of the selected ultrasound images and/or cine clips. Upon the operator of the ultrasound system 10 selecting or clicking the button 148 the process for sharing the selected ultrasound images and/or cine clips ceases or stops.

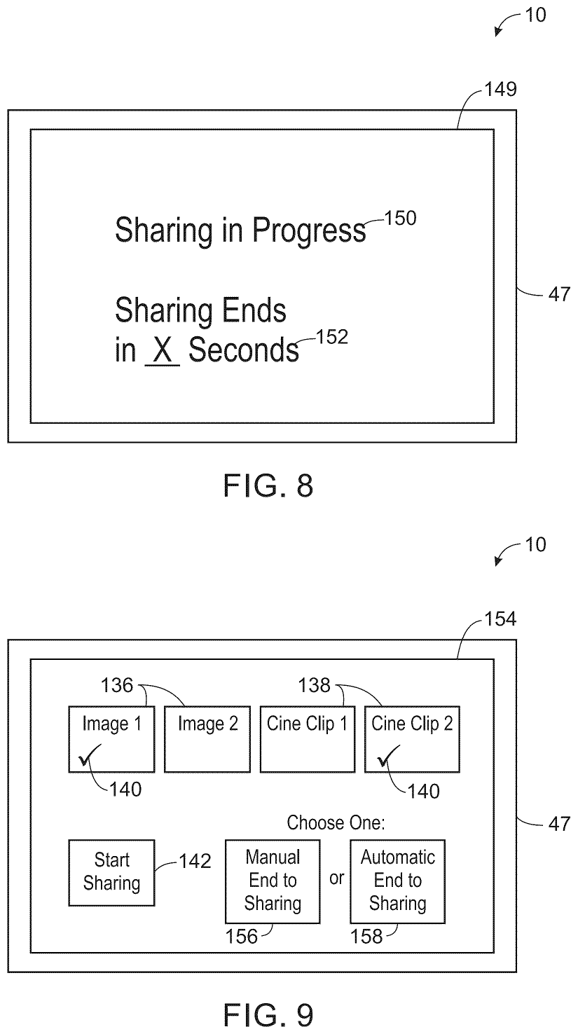

FIG. 8 is a schematic diagram of a user interface 149 on the display of the ultrasound system 10 once sharing of ultrasound imaging data has begun (e.g., having a timer for sharing). In certain embodiments, during sharing of selected ultrasound images and/cine clips an indicator 150 (e.g., text) may be provided on the user interface 149 that sharing the selected ultrasound images and/or cine clips are being shared (e.g., to the portable computing device of the patient just scanned by the ultrasound system 10 or a guardian of the patient when the patient is a child). As depicted, the user interface 149 includes an indicator 152 of an amount of time until the sharing of the selected ultrasound images and/or cine clips automatically stops or ceases.

FIG. 9 is a schematic diagram of a user interface 154 on the display 47 of the ultrasound system 10 for initiating sharing of ultrasound imaging data (e.g. having selection for ending sharing). Similar to user interface 134 in FIG. 6 and as depicted in FIG. 9, the plurality of ultrasound images 136 and cine clips 138 are displayed on the user interface 154 for selection by the operator of the ultrasound system 10. In certain embodiments, the plurality of ultrasound images 136 and cine clips 138 are displayed as thumbnails. Those ultrasound images 136 and cine clips selected (e.g., via a console of the ultrasound system 10) for direct sharing from the ultrasound system 10 to a portable computing device of the patient just scanned with the ultrasound system (or a guardian of the patient when the patient is a child) are marked with an indicator 140. As depicted, the user interface 134 includes a button 142 for selection to initiate the direct sharing of the selected ultrasound images 136 and cine clips 138. Upon the operator of the ultrasound system 10 selecting or clicking the button 142 the process for sharing the selected ultrasound images 136 and cine clips 138 begins. In addition, the user interface 154 provides two buttons 156 and 158 to choose from for determining how to end the sharing of the selected ultrasound images 136 and cine clips 138. Selecting or clicking of the button 156 by the operator results in ending the sharing of the selected ultrasound images 136 and cine clips 138 by the operator clicking a button (e.g., button 148 on the user interface 144 in FIG. 7). Selecting or clicking of the button 156 by the operator results in ending the sharing of the selected ultrasound images 136 and cine clips 138 automatically once a fixed time passes. The selection of button 156 and 158 should occur prior clicking the button 142. Button 142 may be not able to be selected until one of the buttons 156 and 158 is selected. In certain embodiments, upon selecting the button 158 a field may be presented for the operator to enter the desired time for the sharing of the ultrasound images 136 and cine clips 138 before automatically ceasing the sharing. In certain embodiments, the desired time may be selected via a drop-down list.

FIG. 10 is a block diagram of an example processor platform 160 for a portable computing device to be utilized with the disclosed techniques. The processor platform 160 of the illustrated example includes a processor 162. The processor 162 of the illustrated example is hardware. For example, the processor 162 can be implemented by integrated circuits, logic circuits, microprocessors or controllers from any desired family or manufacturer.

The processor 162 of the illustrated example includes a local memory 164 (e.g., a cache). The example processor 162 of FIG. 10 executes the instructions of at least FIGS. 4 and 5 to implement the systems, infrastructure, displays, and associated methods of FIGS. 4 and 5. The processor 162 of the illustrated example is in communication with a main memory including a volatile memory 166 and a non-volatile memory 168 via a bus 170. The volatile memory 166 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 168 may be implemented by flash memory and/or any other desired type of memory device. Access to the main memory 166, 168 is controlled by a clock controller.

The processor platform 160 of the illustrated example also includes an interface circuit 172. The interface circuit 172 may be implemented by any type of interface standard, such as an Ethernet interface, a universal serial bus (USB), and/or a PCI express interface.

In the illustrated example, one or more input devices 174 are connected to the interface circuit 172. The input device(s) 174 permit(s) a user to enter data and commands into the processor 162. The input device(s) 174 can be implemented by, for example, a sensor, a microphone, a camera (still or video, RGB or depth, etc.), a keyboard, a button, a mouse, a touchscreen, a track-pad, a trackball, isopoint and/or a voice recognition system.

One or more output devices 176 are also connected to the interface circuit 172 of the illustrated example. The output devices 176 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 172 of the illustrated example, thus, typically includes a graphics driver card, a graphics driver chip or a graphics driver processor.

The interface circuit 172 of the illustrated example also includes a communication device such as a transmitter, a receiver, a transceiver, a modem and/or network interface card to facilitate exchange of data with external machines (e.g., computing devices of any kind) via a network 178 (e.g., an Ethernet connection, a digital subscriber line (DSL), a telephone line, coaxial cable, a cellular telephone system, Wi-Fi, etc.).

The processor platform 160 of the illustrated example also includes one or more mass storage devices 180 for storing software and/or data. Examples of such mass storage devices 180 include floppy disk drives, hard drive disks, compact disk drives, Blu-ray disk drives, RAID systems, and digital versatile disk (DVD) drives.

Coded instructions 182 may be stored in the mass storage device 180, in the volatile memory 166, in the non-volatile memory 168, and/or on a removable tangible computer readable storage medium (e.g., mass storage device 180).

Technical effects of the disclosed embodiments include providing direct device (e.g., ultrasound system or console) to device (e.g., patient device) connectivity for downloading or sharing images and/or cines on the patient device in a fast, secure, and easy process. Technical effects of the disclosed embodiments also include enabling patients to receive their ultrasound images or cines directly after the scan on their device (e.g., smartphone, tablet, etc.). Technical effects of the disclosed embodiments further include providing the images or cines without any preparations needed on the patient's side (e.g., no dedicated application is needed, ultrasound console works out-of-the box with the operating system of the patient's device, machine-to-machine connection utilized with no Internet or cloud connectivity involved). Technical effects of the disclosed embodiments even further includes enabling image sharing to occur in areas without Internet access on the patient's device (e.g., lower levels of hospitals, rural areas, etc.). Technical effects of the disclosed embodiments yet further include enabling a patient to instantly share or postprocess the image further on their device.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for sharing ultrasound imaging data, comprising:
receiving, at a processor of a console of an ultrasound imaging system, a selection of one or more ultrasound images and/or cine clips of a region of a subject acquired by the ultrasound imaging system;
receiving, at the processor, a first input to start sharing the one or more ultrasound images and/or cine clips to a portable computing device associated with the subject, wherein the portable computing device comprises a camera; and
initiating, via the processor, sharing of the one or more ultrasound images and/or cine clips directly from the console to the portable computing device via a device-to-device connection.

2. The computer-implemented method of claim 1, further comprising ceasing, via the processor, the sharing of the one or more ultrasound images and/or cine clips to the portable computing device.

3. The computer-implemented method of claim 2, wherein ceasing the sharing of the one or more ultrasound images and/or cine clips comprises receiving a second input, at the processor, to cease the sharing of the one or more ultrasound images and/or cine clips.

4. The computer-implemented method of claim 2, wherein ceasing the sharing of the one or more ultrasound images and/or cine clips comprises automatically ceasing, via the processor, the sharing of the one or more ultrasound images and/or cine clips after a fixed amount of time passes after initiation of the sharing of the one or more ultrasound images and/or cine clips.

5. The computer-implemented method of claim 1, further comprising initiating, via the processor, a secure Wi-Fi hotspot for a single inbound connection upon receiving the first input.

6. The computer-implemented method of claim 5, further comprising causing, via the processor, display of a Wi-Fi access quick response code on a display of the ultrasound imaging system, wherein the Wi-Fi access quick response code is natively configured to work with an operating system of the portable computing device.

7. The computer-implemented method of claim 6, further comprising detecting, via the processor, a connection to the secure Wi-Fi hotspot by the portable computing device when the portable computing device utilizes the Wi-Fi access quick response code.

8. The computer-implemented method of claim 7, further comprising initiating, via the processor, a content file server upon detecting the connection to the secure Wi-Fi hotspot, wherein the content file server is restricted to only access the one or more ultrasound images and/or cine clips that were selected.

9. The computer-implemented method of claim 8, further comprising causing, via the processor, display of a content uniform resource locator quick response code on the display of the ultrasound imaging system, wherein the content uniform resource locator quick response code is natively configured to work with an operating system of the portable computing device, and the content uniform resource locator quick response code is configured to provide the portable computing device access to the one or more ultrasound images and/or cine clips when utilized by the portable computing device.

10. An ultrasound imaging system, comprising:
a display;
a memory encoding processor-executable routines; and
a processor configured to access the memory and to execute the processor-executable routines, wherein the processor-executable routines, when executed by the processor, cause the processor to:
receiving a selection of one or more ultrasound images and/or cine clips of a region of a subject acquired by the ultrasound imaging system;
receiving a first input to start sharing the one or more ultrasound images and/or cine clips to a portable computing device associated with the subject, wherein the portable computing device comprises a camera; and
initiating sharing of the one or more ultrasound images and/or cine clips directly from the ultrasound imaging system to the portable computing device via a device-to-device connection.

11. The ultrasound imaging system of claim 10, wherein the processor-executable routines, when executed by the processor, further cause the processor to cease the sharing of the one or more ultrasound images and/or cine clips to the portable computing device.

12. The ultrasound imaging system of claim 11, wherein ceasing the sharing of the one or more ultrasound images and/or cine clips comprises receiving a second input to cease the sharing of the one or more ultrasound images and/or cine clips.

13. The ultrasound imaging system of claim 11, wherein ceasing the sharing of the one or more ultrasound images and/or cine clips comprises automatically ceasing the sharing of the one or more ultrasound images and/or cine clips after a fixed amount of time passes after initiation of the sharing of the one or more ultrasound images and/or cine clips.

14. The ultrasound imaging system of claim 10, wherein the processor-executable routines, when executed by the processor, further cause the processor to initiate a secure Wi-Fi hotspot for a single inbound connection upon receiving the first input.

15. The ultrasound imaging system of claim 14, wherein the processor-executable routines, when executed by the processor, further cause the processor to cause display of a Wi-Fi access quick response code on the display of the ultrasound imaging system, wherein the Wi-Fi access quick response code is natively configured to work with an operating system of the portable computing device.

16. The ultrasound imaging system of claim 15, wherein the processor-executable routines, when executed by the processor, further cause the processor to detect a connection to the secure Wi-Fi hotspot by the portable computing device when the portable computing device utilizes the Wi-Fi access quick response code.

17. The ultrasound imaging system of claim 16, wherein the processor-executable routines, when executed by the processor, further cause the processor to initiate a content file server upon detecting the connection to the secure Wi-Fi hotspot, wherein the content file server is restricted to only access the one or more ultrasound images and/or cine clips that were selected.

18. The ultrasound imaging system of claim 17, wherein the processor-executable routines, when executed by the processor, further cause the processor to cause display of a content uniform resource locator quick response code on the display of the ultrasound imaging system, wherein the content uniform resource locator quick response code is natively configured to work with an operating system of the portable computing device, and the content uniform resource locator quick response code is configured to provide the portable computing device access to the one or more ultrasound images and/or cine clips when utilized by the portable computing device.

19. A non-transitory computer-readable medium, the computer-readable medium comprising processor-executable code that when executed by a processor, causes the processor to:
receiving a selection of one or more ultrasound images and/or cine clips of a region of a subject acquired by the ultrasound imaging system;
receiving a first input to start sharing the one or more ultrasound images and/or cine clips to a portable computing device associated with the subject, wherein the portable computing device comprises a camera; and
initiating sharing of the one or more ultrasound images and/or cine clips directly from the ultrasound imaging system to the portable computing device via a device-to-device connection.

20. The non-transitory computer-readable medium of claim 19, wherein the processor-executable code, when executed by the processor, further causes the processor to cease the sharing of the one or more ultrasound images and/or cine clips to the portable computing device.
